(19) United States
(12) Patent Application Publication
(10) Pub. No.: US 2010/0034959 A1
(43) Pub. Date: Feb. 11, 2010

(54) HIGH SOLIDS, HIGH MOLECULAR WEIGHT POLYMER COATING

(75) Inventors: BRIAN K. JENSEN, Cedar Rapids, IA (US); SHAWN M. ENGELS, Tipton, IA (US)

Correspondence Address: MCKEE, VOORHEES & SEASE, P.L.C., 801 GRAND AVENUE, SUITE 3200, DES MOINES, IA, 50309-2721, US

(73) Assignee: VECTOR CORPORATION, Marion, IA (US)

(21) Appl. No.: 12/512,433

(22) Filed: Jul. 30, 2009

Related U.S. Application Data
(60) Provisional application No. 61/087,086, filed on Aug. 7, 2008.

(51) Int. Cl. A61K 9/28; C09D 101/08; C09D 103/02; C09D 129/10

(52) U.S. Cl. 427/2.18; 427/2.14; 106/163.01; 106/205.01; 106/287.26

(57) ABSTRACT

A method of coating tablets with a high molecular weight polymer wherein the high molecular weight polymer is suspended in a non-solvent, non-aqueous liquid carrier. The liquid carrier may include a low-viscosity binder. Preferably, the binder is included in concentrations of around 1-10% by weight in solution in the carrier. The high molecular weight polymer may be micronized prior to its suspension in the liquid carrier. Once suspended, the liquid is introduced into a spray coating machine such as a partially or fully perforated pan system wherein the tablets are coated as desired.

### CROSS-REFERENCE TO RELATED APPLICATIONS

This application claims priority under 35 U.S.C. §119 of a provisional application Ser. No. 61/087,086 filed Aug. 7, 2008, which application is hereby incorporated by reference in its entirety.

### BACKGROUND OF THE INVENTION

High molecular weight polymers are used to coat tablets and incorporated into tablet formulas for a variety of reasons. These reasons can include gastric retention, mucoadhesion, sustained release of the medication and various combinations of these applications. Generally, the high molecular weight polymer coating is either incorporated into the tablet formula or is applied onto the exterior of the tablet.

Incorporating a high molecular weight polymer coating into the tablet formula requires a relatively large amount of high molecular weight polymer coating to perform as desired. This relatively large amount of high molecular weight polymer coating in the tablet formula forces the tablet to be much larger than simply coating the exterior of the tablet.

Currently, coating the exterior of the tablet requires either a tablet press or a spray coating application. A tablet press is expensive and inflexible. For example, a tablet press for a circular shaped tablet will not work for an oval shaped tablet. This requires purchasing of new equipment for new shapes or limits the manufacturer's ability to shape their tablets as desired. Alternatively, spray coating a high molecular weight polymer coating onto a tablet is also currently fraught with problems.

With the conventional spray polymer layering process, the polymers must be soluble so as to dissolve in a suitable solvent so as to be applied as a dilute liquid. Typical soluble polymers will be 5-15% solids in solution, by weight. In a pharmaceutical application, the polymers may function for modified release of active ingredients and/or for taste masking. In this type of application, polymers may be layered on the cores for 5-25% weight gain. In the case of organic solvent soluble polymers, as much as 5 kg of solvent must be used for each 1 kg of product coated. In scaled production, this is a very large amount of solvent per coated batch. For example, in a 5 kg batch of cores coated to a 25% weight gain with a 5% solids solution, a 25 kg solution is required, with the polymer application being approximately 2.5 grams of polymer substances per minute. Thus, the conventional layering process with dissolved polymers in solvent solution is slow and requires large volumes of solvents.

If polymer content in solution is not kept very low as described above, the viscosity of the solution becomes unprocessable and coating defects increase the number of rejected tablets to unacceptable levels. Some high molecular weight polymer coatings even have unacceptable viscosities at very low solution concentrations. It is therefore desirable to provide a method of coating tablets with a high molecular weight polymer coating which avoids the problems of the prior art.

Accordingly, a primary objective of the present invention is the provision of a method of applying a high molecular weight polymer coating to a tablet which overcomes the problems in the prior art.

Another objective of the present invention is the provision of a method of applying a high molecular weight polymer coating to a tablet which allows a variety of tablet shapes and sizes to be coated without the need for specially shaped tablet presses.

Still another objective of the present invention is a method of applying a high molecular weight polymer coating to a tablet which allows for spray application of the coating without the need for high volumes of solution.

Another objective of the present invention is the provision of a method of applying a high molecular weight polymer coating to a tablet which allows for the rapid deposition of the coating in a manner which still results in conformal tablets without unacceptably high numbers of rejected tablets.

Yet another objective of the present invention is a method of applying a high molecular weight polymer coating to a tablet which is efficient and economical.

These and other objectives will become apparent from the following description of the invention.

### SUMMARY OF THE INVENTION

Generally, in the method of coating tablets with a high molecular weight polymer of the present invention, the high molecular weight polymer is suspended in a liquid carrier to which the polymer is insolvent. The liquid carrier may include a low-viscosity binder. Preferably, the binder is included in concentrations of around 1-10% by weight in solution in the carrier. The high molecular weight polymer may be micronized prior to its suspension in the liquid carrier. Once suspended, the liquid is introduced into a spray coating machine such as a partially or fully perforated pan system wherein the tablets are coated as desired.

### DETAILED DESCRIPTION OF THE PREFERRED EMBODIMENT

The present invention will be described as it applies to its preferred embodiment. It is not intended that the present invention be limited to the described embodiment. It is intended that the invention cover all modifications and alternatives which may be included within the spirit and scope of the invention.

For purposes of the present invention, a high molecular weight polymer is any whose viscosity, if used in solution, is greater than 45 centipoise at 2% w/w concentration in said solution. Common high molecular weight polymer coatings include, but are not limited to, hydroxypropyl cellulose, hydroxypropylmethyl cellulose, ethyl cellulose, poly ethylene oxide, and certain natural gums (guar, locust bean, etc). Preferably, the high molecular weight polymer is micronized, meaning the particle size is generally below 100 microns in maximum diameter and preferably is less than 20 microns in average diameter, but generally not less than 1 micron in average diameter.

A high molecular weight polymer coating, such as any of those discussed above, is preferably applied to a tablet using a liquid carrier. Water is not an acceptable carrier as most high molecular weight polymer coatings available today are water soluble. While the carrier may be a solvent for other substances, a preferred carrier is a liquid in which the selected high molecular weight polymer coating is insoluble. For example, anhydrous ethanol is a suitable carrier when used with polyethylene oxide.

Anhydrous Ethanol is also good for Hydroxypropulmethyl and cellulose gum. Other nonsolvents include glycerin, aliphatic and aromatic solvents for Hydroxypropyl Cellulose. Poly Ethylene Oxide, Hydroxypropylmethyl, and Hydroxypropyl Cellulose are the leaders in high molecular weight film forming. Some of the natural gums also can work, and tend to be insoluble in anhydrous ethanol also.

Any carrier must be a non-aqueous (not water), non-solvent liquid. By using such carriers, the high molecular weight polymer coating is not able to dissolve or build any appreciable viscosity which allows for very high loadings in the suspension.

A small amount of a binder may be added to the carrier liquid. Such binders include, but are not limited to, polyvinylpyrrolidone K-30 or K-90, hydroxypropyl cellulose, hydroxypropylmethyl cellulose (hypromellose) (E-5 and E-15 should not be used in an anhydrous ethanol system) or any relatively lower molecular weight polymer or other soluble material such as a resin, sugar, dextrins, maltodextrins, starches, lignins, etc. Preferably, the binder is added in concentrations of about 1-10% by weight in solution in the carrier. The use of a binder is the preferred method of adhering the coating to the tablet and eliminates the need to use a pH modification.

Generally, once an amount of carrier is selected, then a corresponding amount of binder may be used. Preferably, the carrier is stirred to form a vortex. Slowly, the binder is added and allowed to completely dissolve. An additional emulsifier may be used if desired. The high molecular weight polymer coating is then added to the carrier liquid and suspended by mixing for a sufficient amount of time. The suspension should appear uniform.

Once the high molecular weight coating is suspended in the carrier liquid, the tablets may be coated. Preferably, the tablets are added to a coating machine such as a standard perforated pan system known as the Vector Hi-Coater made by Vector Corporation. Other coating machines, including fluidized bed machines, Wurster-style coating machines, rotating bed coating machines, and others may be used.

In this example, a laboratory development coating system such as the LDCS Pilot Hi-Coater made by Vector Corporation is used. Ethanol is selected as the carrier. PVP K-30 was selected as the binder and polyethylene oxide is used as the high molecular weight polymer coating.

To prepare the solution, pour 950 g of 200 proof ethanol into a beaker. Place the beaker into a mixer and turn on the mixer to create a vortex in the ethanol. Slowly add 50 g of PVP K-30 powder while continuing to mix. Allow the PVP to completely dissolve. Once dissolved, add 1 g of Tween 80 and allow the mixture to continue to stir for five minutes. Next, add 200 g of polyethylene oxide which has been micronized to the solution and allow it to continue to mix for 30 minutes. After 30 minutes, the suspension should be white and uniform in appearance.

Once the suspension has been prepared, load 800 g of tablets into the 1.5 L pan of the LDCS. Warm the tablet bed up to an exhaust temperature of 30° C. while jogging the pan at 5 revolutions per minute. Begin to spray the suspension onto the tablets once the pan speed has reached 25 revolutions per minute, the airflow is at 60 cubic feet per minute, the pump speed is at 25 revolutions per minute. Nozzle air should be set at 13 pounds per square inch and the inlet air temperature should be around 45° C. with exhaust air temperature at 25-30° C. Coating should be applied until the desired weight gain has been achieved. Then the tablets should be tumbled at 10 revolutions per minute to dry for 30 minutes. Once dry, the tablets can be discharged.

The invention has been shown and described above with the preferred embodiments, and it is understood that many modifications, substitutions, and additions may be made which are within the intended spirit and scope of the invention. From the foregoing, it can be seen that the present invention accomplishes at least all of its stated objectives.

What is claimed is:

1. A method of coating tablets with a high molecular weight polymer, the method comprising:
obtaining an amount of high molecular weight polymer;
suspending at least a portion of the high molecular weight polymer in a non-solvent, non-aqueous carrier liquid to form a mixture; and
spraying the mixture onto tablets so as to form a coating on a tablet.

2. The method of coating tablets with a high molecular weight polymer of claim 1 further comprising introducing a binder into the carrier liquid.

3. The method of coating tablets with a high molecular weight polymer of claim 2 wherein the binder is in concentrations of 1-10% by weight of the carrier liquid.

4. The method of coating tablets with a high molecular weight polymer of claim 1 wherein the mixture is sprayed onto the tablets through the use of a rotor coating machine.

5. The method of coating tablets with a high molecular weight polymer of claim 1 wherein the mixture is sprayed onto the tablets through the use of a Wurster-style coating machine.

6. The method of coating tablets with a high molecular weight polymer of claim 1 wherein the mixture is sprayed onto the tablets through the use of a perforated pan coating machine.

7. The method of coating tablets with a high molecular weight polymer of claim 1 wherein the high molecular weight polymer is micronized prior to its suspension in the carrier.

8. The method of coating tablets with a high molecular weight polymer of claim 1 wherein the carrier is ethanol.

9. The method of coating tablets with a high molecular weight polymer of claim 1 wherein the high molecular weight polymer is selected from the group consisting of:
hydroxypropyl cellulose;
hydroxypropylmethyl cellulose;
cellulose gum;
poly ethylene oxide; and
a natural gum.

10. A mixture for coating tablets, the mixture comprising:
a high molecular weight polymer suspended in a non-solvent, non-aqueous carrier liquid; and
a binder.

11. The mixture for coating tablets of claim 10 wherein the binder is 1-10% by weight of the mixture.

12. The mixture for coating tablets of claim 10 wherein the high molecular weight polymer is micronized.

13. The mixture for coating tablets of claim 10 wherein the high molecular weight polymer is selected from the group consisting of: hydroxypropyl cellulose;
hydroxypropylmethyl cellulose;
cellulose gum;
poly ethylene oxide; and
a natural gum.
